(19) United States
(12) Patent Application Publication
(10) Pub. No.: US 2005/0232956 A1
(43) Pub. Date: Oct. 20, 2005

(54) Method for separating saturated and unsaturated fatty acid esters and use of separated fatty acid esters

(76) Inventors: Shailendra Bist, Torrance, CA (US); Bernard Y. Tao, Lafayette, IN (US)

Correspondence Address: BAKER & DANIELS LLP, 300 NORTH MERIDIAN STREET, SUITE 2700, INDIANAPOLIS, IN, 46204, US

(21) Appl. No.: 11/068,104

(22) Filed: Feb. 28, 2005

Related U.S. Application Data
(60) Provisional application No. 60/547,992, filed on Feb. 26, 2004.

(51) Int. Cl. A61K 7/00; C07C 51/43; C10L 1/18

(52) U.S. Cl. 424/401; 44/385; 554/186

(57) ABSTRACT

A method for treating a fatty acid methyl ester. The method can include mixing the fatty acid methyl ester with an amount of urea and an amount of alcohol to make (i) a urea/fatty acid methyl ester ratio of from about 0 wt/wt to about 1 wt/wt and (ii) an alcohol/fatty acid methyl ester ratio of from about 4 vol/wt to about 8 vol/wt, heating the fatty acid methyl ester/urea/alcohol mixture to a temperature at which a homogenous mixture is obtained, cooling the fatty acid methyl ester/urea/alcohol mixture to a temperature where a solid phase and a liquid phase are formed, and separating the solid phase from the liquid phase.

### TECHNICAL FIELD OF THE DISCLOSURE

The present invention generally relates to a method of processing fatty acids. The present invention particularly relates to a method for separating saturated and unsaturated fatty acids. Separated fractions of fatty acid esters are useful as fuels.

### BACKGROUND OF THE DISCLOSURE

Urea is known to form inclusion complexes with long chain organic compounds. This was first discovered and reported by F. Bengen in a German patent filed in 1940. Later studies from the late forties to the early fifties reported the selectivity of urea in forming complexes with long chain organic molecules. This selectivity was found to be based on; a) Carbon chain length, b) presence of unsaturation in the molecule, and c) degree of unsaturation. The formation of these complexes was found to be a powerful technique for the separation of a mixture of saturated and unsaturated organic compounds, e.g. fractionation of a mixture of free fatty acids. Various techniques for the formation of such complexes were also studied, however with little or no focus on the process parameters. Work done by Hayes et al., on the fractionation of fatty acids studied various process parameters that effect the formation of urea inclusion complexes, the product yields, and the composition of fractions obtained. Patents U.S. Pat. No. 5,106,542, U.S. Pat. No. 5,243,046 describe the art of fractionating fatty acid mixtures via urea inclusion. U.S. Pat. No. 5,679,809 describes the concentration of polyunsaturated fatty acid ethyl esters via urea inclusion.

Fatty acid esters find a variety of uses including in foodstuffs, nutritive compositions, pharmaceuticals, cosmetics, dermatological compositions, and drying oils for coatings and paints.

Biodiesel, according to ASTM D-6751 specification, is defined as a fuel comprised of mono-alkyl esters of long chain fatty acids derived from vegetable oils or animal fats. The alkyl group is of the type CₙH₂ₙ₊₁, preferable methyl (CH₃), the oil source is preferable soybean. Biodiesel derived from a soy oil source is referred to as SME (soy methyl esters) or soy biodiesel, EtOH refers to ethanol, and urea. elsewhere in the text.

### REFERENCES

1. Bengen, F., German Patent Application O. Z. 12438, Mar. 18, 1940.
2. Swern, D. “Urea and Thiourea Complexes in Separating Organic Compounds,” Industrial and Engineering Chemistry, Vol. 47, 216-221, 1955.
3. Swern, D., Parker, W. E., “Application of Urea Complexes in the Purifcation of Fatty Acids, Estes, and Alcohols. 1. Oleic Acid from Inedible Animal Fats,” JAOCS, 431-434, 1952.
4. Newey, H. A., Shokal, E. C., Mueller, A. C., Bradley, T. F., “Industrial and Engineering Chemistry,” Vol. 42, 2538-2540, 1950.
5. Schlenk, H., Holman, R. T., “Separation and Stabilization of Fatty Acids by Urea Complexes,” Journal of American Chemical Society, vol. 72, 5001-5005, 1950.
6. Hayes, D. G., Bengtsson, Y. C., Alstine, J. M. V., Setterwall, F., “Urea Complexation for the Rapid, Ecologically Responsible Fractionation of Fatty Acids from Seed Oil,” vol. 75, JAOCS, 103-1409, 1998.
7. Hayes, D. G., Bengtsson, Y. C., Alstine, J. M. V., Setterwall, F., “Urea-Based Fractionation of Seed Oil Samples Containing Fatty Acids and Acylglycerols of Polyunsaturated and Hydroxy Fatty Acids,” Vol. 77, JAOCS, 207-213, 2000.
8. Hayes, D. G., “Free Fatty Acid Fractionation via Urea Inclusion Compounds,” Vol. 13, INFORM, 832-833, 2002.
9. Hayes, D. G., Alstine, J. M. V., Asplund, A. L., “Triangular Phase Diagrams to Predict the Fractionation of Free Fatty Acid Mixtures via Urea Complex Formation,” Separation Science and Technology, Vol. 36, 45-58, 2001.
10. Lee, L. A. Johnson and E. G. Hammond, “Reducing the Crystallization Temperature of Biodiesel by Winterizing Methyl Soyate,” JAOCS, Vol. 73, No. 5 (1996).
11. R. O. Dunn, M. W. Shockley, and M. O. Bagby, “Improving the Low-Temperature Properties of Alternative Diesel Fuels: Vegetable Oil-Derived Methyl Esters,” JAOCS, Vol. 73, No. 12 (1996).
12. Diks, R. M. M., Lee, M. J., “Production of Very Low Saturate Oil Based on the Specificity of Geotrichum Candidum Lipase,” JAOCS, Vol. 76, No. 4, 1999.
13. Shimada, Y., Maruyama, K., Okazaki, S., Nakamura, M., Sugihara, C., “Enrichment of Polyunsaturated fatty Acids with Geotrichum Candidum Lipase,” JAOCS, Vol. 71, 951-953, 1994.
14. U.S. Pat. No. 5,678,809, “Concentration of Polyunsaturated Fatty Acid Ethyl Esters and Preparation Thereof”.
15. U.S. Pat. No. 5,106,542, “Process for the Continuous Fractionation of a Mixture of Fatty Acids”.
16. U.S. Pat. No. 5,243,046, “Process for the Continuous Fractionation of a Mixture of Fatty Acids”.
17. U.S. Pat. No. 6,444,784 B1, “Wax Crystal Modifiers”.
18. U.S. Pat. No. 6,409,778 B1, “Additive for Biodiesel and Biofuels”.
19. International Publication No. WO 99/62973, “Wax Crystal Modifiers Formed Form Dialkyl Phenyl Fumarate”
20. International Publication No. WO 00/32720, “Winterized Paraffin Crystal Modifiers”
21. U.S. Pat. No. 3,961,916, “Middle Distillate Composition with Improved Filterability and Process Thereof”
22. U.S. Pat. No. 5,726,048, “Mutant of Geotricum Candidum Which Produces Novel Enzyme System to Selectively Hydrolyze Triglycerides”.
23. U.S. Pat. No. 6,537,787, “Enzymatic Methods for Polyunsaturated Fatty Acid Enrichment”
24. U.S. Pat. No. 5,470,741, “Mutant of Geotrichum Candidum Which Produces Novel Enzyme System to Selectively Hydrolyze Triglycerides”
25. Kocherginsky et al., “Mass Transfer of Long Chain Fatty Aids Through Liquid-Liquid Interface Stabilized by Porous Membrane,” Separation Purification Technology, Vol. 20, 197-208, 2000.
26. U.S. Pat. No. 4,542,029, “Process for Separating Fatty Acids”
27. U.S. Pat. No. 4,049,688, “Process for Separating Esters of Fatty Acids by Selective Adsorption”
28. U.S. Pat. No. 4,129,583, “Process for Separating Crystallizable Fractions From Mixtures Thereof
29. Maeda, K., Nomura, Y., Tai K., Uneo, Y., Fukui, K., Hirota, S., “New Crystallization of Fatty Acids From Aqueous Ethanol Solution Combined with Liquid-Liquid Extraction,” Ind. Eng. Chem. Res., Vol. 38, 2428-2433, 1999.

All of the above references are incorporated herein by reference.

Methods in the art for fractionating SME to improve its cold flow properties are based on thermal crystallization (winterization) followed by filtration (with or without solvent). Both techniques rely on the difference in crystallization temperature of the saturated and unsaturated components of SME. The saturates crystallize at a higher temperature and can be removed via filtration, centrifugation etc. However, due to co-crystallization of the components significant amounts of unsaturates are also removed, resulting in high losses. For a C.P. of −16 C almost 75% of the starting material was removed in work done by Dunn et al. These techniques involve cooling to very low temperatures and process time running into days. It would be advantageous to identify a method for separation of fatty acid methyl esters applicable to industrial scale application.

### SUMMARY OF THE DISCLOSURE

This invention relates to the fractionation/separation of fatty acid methyl esters, specifically SME into saturated fatty acid-rich and unsaturated fatty acid-rich fractions via the use of urea inclusion/urea complexation. Operation of diesel engines using renewable energy sources including soy bean derived fuels is known, as is the challenge of overcoming negative properties of soy derived fuels, e.g., the gelling of bioderived diesel (biodiesel) at higher temperatures than petroleum derived fuels. The composition of biodiesel (for a typical sample of soy biodiesel) is as given in table (1).

TABLE 1

| Fatty Acid Methyl Ester | % by Weight |
|---|---|
| Methyl Palmitate (C16:0) | 10.3 |
| Methyl Sterate (C18:0) | 4.7 |
| Methyl Oleate (C18:1) | 22.5 |
| Methyl Linoleate (C18:2) | 54.1 |
| Methyl Linoleniate (C18:3) | 8.3 |

The present invention includes one or more of the following features: A very controlled C.P. depression can be achieved ranging from about 2 to about 26 C°. ‘Cloud point depression’ is the difference in C.P. of the product and the starting material. By controlling three process parameters the process can be optimized for maximum efficiency. Here efficiency of the process is in terms of the highest yield achieved for a given C.P. drop. Low processing cost, short processing time, easily scalable and robust process. A robust process is a process that is reproducible and repeatable with negligible variation in the results. Low energy consumption. Raw materials can be recycled and reused. The process is ecologically friendly with all raw materials, intermediates and final products and wastes being biodegradable. Applicable to the fractionation of SME, specifically soy derived biodiesel. Applicable to the fractionation of any mixture of vegetable oil derived free fatty acid methyl esters. It could be seen as altering the composition of SME for our benefit, than just separating the saturated and unsaturated fractions. It could be seen as altering the composition of any mixture of vegetable oil derived free fatty acid methyl esters to our benefit, rather than just separating the saturated and unsaturated fractions. It could be seen as an efficient method of obtaining an unsaturate rich and a saturate rich fraction from any mixture of vegetable oil derived free fatty acid methyl esters.

The novel method described herein selectively removes saturate fatty acid-rich fractions from SME. The amount of saturates removed and hence the C.P. of the resulting product can be easily controlled. We have achieved C.P. of about −10 C° at a yield of 78.38% and −26 C° at a yield of 66.39% by weight of the starting material. The novel process requires only moderate temperature variations (20-75 C°). The raw materials used may be recovered and reused making the process compatible with the environment. The processing time is short and runs into several minutes. This makes the process ideal for industrial scale processing of SME for improving its cold flow properties. In another embodiment our invention provides a very efficient and effective means of separation of unsaturated-rich and saturated-rich fractions from any mixture of vegetable oil derived free fatty acid methyl esters. In yet another embodiment our invention provides an efficient and effective means of altering the composition of any mixture of vegetable oil derived free fatty acid methyl esters. Specifically altering the ratio of saturates to unsaturates in a mixture. The present invention helps address: Controlled Cloud point depression of SME; controlled cloud point depression of any mixture of vegetable oil derived free fatty acid methyl esters; Efficient separation of unsaturated and saturated fractions from any mixture of vegetable oil derived free fatty acid methyl esters; altering the composition of any mixture of vegetable oil derived free fatty acid methyl esters; specifically altering the ratio of saturates to unsaturates in a mixture.

### DETAILED DESCRIPTION OF THE DISCLOSURE

While the invention is susceptible to various modifications and alternative forms, a specific embodiment thereof has been shown by way of example and will herein be described in detail. It should be understood, however, that there is no intent to limit the invention to the particular form disclosed, but on the contrary, the intention is to cover all modifications, equivalents, and alternatives falling within the spirit and scope of the invention as defined by the appended claims.

SME has proven to be an extender/additive/replacement for diesel fuel, heating oil and studies are on for its development as an aviation turbine fuel extender. A challenge to the utilization of biodiesel is its poor cold flow properties. The total saturate content of about 14-16% (wt/wt) causes the C.P. (cloud point) to be about 0 C° and pour point to be around −2 to −4 C°. This limits the use of SME at low temperatures. Various efforts have been made to reduce or depress the C.P. of SME by: 1) removal of saturated components, 2) use of cold flow additives, 3) use of branched chain alcohol esters, 4) combinations thereof.

The popular method for removal of saturate components is winterizing or cold filtering. Various studies have been conducted, however these methods have very low yields for any significant change in the C.P. Cold flow additives have been successful in lowering the P.P (Pour Point), however have little or no effect on the C.P. of SME. Usage of branched chain alcohol esters have poor yields during the esterification reaction, higher raw material cost with only a small depression in C.P.

We have developed a method for a controlled fractionation/separation of the saturated and unsaturated fractions of SME, using urea inclusion/urea complexation. By controlling three process parameters we achieve a C.P. depression ranging from −2 C° to −26 C°, by a controlled removal of the saturated fatty acid-rich fraction, with unsaturated fatty acid-rich fraction yields ranging from 98%-65% of the starting material, respectively. The three process parameters being 1) urea/SME/Alcohol (weight/weight/volume ratio), 2) cooling temperature, and 3) rate of cooling.

Depending upon the desired C.P. drop, we can choose a combination of urea/SME/Alcohol ratio, cooling temperature and rate of cooling. Various such combinations arc possible for the same C.P. drop. Those three parameters are selected to achieve an optimal fractionation of SME resulting in the highest yield for a given C.P. drop. The urea/SME ratio may range from 0 to 1 wt/wt. The alcohol/SME ratio may range from 4 to 8 vol/wt. Useful alcohols have from 1 to 5 carbon atoms, and blends thereof. Denatured ethanol is demonstrated to be effective.

The urea/SME/alcohol mixture is heated with constant stirring to about 65-75 C°, until a homogenous mixture is reached. The rate of heating of the mixture does not have an effect on the efficiency of the process. Here efficiency of the process is in terms of the yield of the product and the C.P. achieved.

The resulting mixture is then cooled at a preselected rate of cooling, with constant stirring, to a preselected temperature. The rate of cooling may vary from 1 to 15 C°/min. The final temperature to which the mixture is cooled may vary from 10 to 50 C°.

A solid phase comprised of urea inclusion compounds. This phase is rich in saturates. This maybe referred to as Solid phase; urea inclusion compound or raffinate elsewhere in the text.

A liquid phase comprised of mostly unsaturates, alcohol and some dissolved urea. The urea inclusion compounds formed are then removed from the liquid phase via filtration/centrifugation.

50-70% of the starting volume of alcohol is then recovered from the filtrate via evaporation at a temperature between 30-50 C° (under vacuum). The evaporation of ethanol is stopped just before the solubility limit of urea is reached. This prevents the urea coming out of solution and forming further complexes with the dissolved saturate rich fraction of SME. The remaining filtrate is then washed with warm acidic water (60-70 C°, pH 3-4) to remove all the urea and remaining alcohol. This can be done in steps, washing the filtrate with an equal volume of warm, acidified water in each step, or in a continuously manner using water 3-4 four times the volume of filtrate. Unsaturate rich SME fraction with the desired C.P. is then achieved. The saturate rich fraction can also be obtained from the raffinate by dissolving and washing with warn acidified water (60-70 C°, pH 3-4). This fraction can be used as additives to heating oil and other heavy oils where C.P. is not a critical property. Urea can be recovered by evaporating the water.

The invention/technique is illustrated by the following examples:

### EXAMPLE 1

The starting SME had the composition and properties according to Table 2: Table 2:

TABLE 2

| | Percentage by |
|---|---|
| Fatty Acid Methyl Ester | weight composition |
| Methyl Palmitate (C16:0) | 9.15 |
| Methyl Stearate (C18:0) | 3.78 |
| Methyl Oleate (C18:1) | 23.52 |
| Methyl Linoleate (C18:2) | 55.25 |
| Methyl Linoleniate (C18:3) | 7.64 |
| Others | 0.66 |
| Total Saturates | 12.93 |
| Cloud Point: (C.°) | 0 |

24.057 g of soy methyl esters and 10.077 g of urea were added to 160 mL of EtOH and the mixture was heated to 67 C°, with constant stirring. A homogenos mixture was obtained with all the urea dissolving at this temperature. The mixture was then cooled at a rate of 1.19 C°/min to a final temperature of 20 C°. The urea inclusions compounds formed were then separated by filtration. The filtrate was then heated to 30 C° and 70% of the starting volume of EtOH was recovered via evaporation under vacuum. The remaining filtrate was then washed with equal volume of water (60 C°, pH 3). This step was repeated twice. 18.83 g of fractionated SME (78.38% by wt of the starting SME) was recovered with the composition and properties according to Table 3. Recovered EtOH is available for re-use in the process.

TABLE 3

| | Percentage by |
|---|---|
| Fatty Acid Methyl Ester | weight composition |
| Methyl Palmitate (C16:0) | 6.34 |
| Methyl Stearate (C18:0) | 1.39 |
| Methyl Oleate (C18:1) | 24.57 |
| Methyl Linoleate (C18:2) | 59.61 |
| Methyl Linoleniate (C18:3) | 8.07 |
| Others | 0.02 |
| Total Saturates | 7.73 |
| Cloud Point: (C.°) | −10 |

### EXAMPLE 2

The starting SME had the composition and properties according to Table 2:

24.053 g of SME and 18.045 g of urea were added to 160 mL of EtOH and the mixture was heated to 73 C°, with constant stirring. A homogenous mixture was obtained with all the urea dissolving at this temperature. The mixture was then cooled at a rate of 1.19 C°/min to a final temperature of 20 C°. The urea inclusions compounds formed were then separated by filtration. The filtrate was then heated to 30 C° and 52% of the starting volume of EtOH was recovered via evaporation under vacuum. The filtrate was then washed with equal volume of water (60 C°, pH 3). This step was repeated twice. 15.97 g of fractionated SME (66.39% by wt of the starting SME) was recovered with the composition and properties according to Table 4.

TABLE 4

| | Percentage by |
|---|---|
| Fatty Acid Methyl Ester | weight composition |
| Methyl Palmitate (C16:0) | 1.55 |
| Methyl Stearate (C18:0) | 0.00 |
| Methyl Oleate (C18:1) | 21.92 |
| Methyl Linoleate (C18:2) | 69.47 |
| Methyl Linoleniate (C18:3) | 7.03 |
| Others | 0.03 |
| Total Saturates | 1.55 |
| Cloud Point: (C.°) | −26 |

### EXAMPLE 3

The starting SME had the composition and properties according to Table 2:

24.056 g of SME and 16.041 g of urea were added to 160 mL of EtOH and the mixture was heated to 72 C°, with constant stirring. A homogenous mixture was obtained with all the urea dissolving at this temperature. The mixture was then cooled at a rate of 1.32 C°/min to a final temperature of 30 C°. The urea inclusions compounds formed were then separated by filtration. The filtrate was then heated to 30 C° and 63% of the starting volume of EtOH was recovered via evaporation under vacuum. The filtrate was then washed with equal volume of water (60 C°, pH 3). This step was repeated twice. 18.25 g of fractionated SME (75.86% by wt of the starting SME) was recovered with the composition and properties according to Table 5.

TABLE 5

| | Percentage by |
|---|---|
| Fatty Acid Methyl Ester | weight composition |
| Methyl Palmitate (C16:0) | 2.25 |
| Methyl Stearate (C18:0) | 0.00 |
| Methyl Oleate (C18:1) | 22.45 |
| Methyl Linoleate (C18:2) | 68.53 |
| Methyl Linoleniate (C18:3) | 6.75 |
| Others | 0.02 |
| Total Saturates | 2.25 |
| Cloud Point: (C.°) | −16 |

### EXAMPLE 4

The starting SME had the composition and properties in Table 2:

24.089 g of SME and 16.044 g of urea were added to 160 ml of EtOH and the mixture was heated to 72 C°, with constant stirring. A homogenous mixture was obtained with all the urea dissolving at this temperature. The mixture was the cooled at a rate of 10.71 C°/min to a final temperature of 20 C°. The urea inclusions compounds formed were then separated by filtration. The filtrate was then heated to 30 C° and 63% of the starting volume of EtOH was recovered via evaporation under vacuum. The filtrate was then washed with equal volume of water (60 C°, pH 3). This step was repeated twice. 15.64 g of fractionated SME (64.92% by wt of the starting SME) was recovered with the composition and properties in Table 6.

TABLE 6

| | Percentage by |
|---|---|
| Fatty Acid Methyl Ester | weight composition |
| Methyl Palmitate (C16:0) | 2.08 |
| Methyl Stearate (C18:0) | 0.00 |
| Methyl Oleate (C18:1) | 24.04 |
| Methyl Linoleate (C18:2) | 66.03 |
| Methyl Linoleniate (C18:3) | 7.54 |
| Others | 0.01 |
| Total Saturates | 2.08 |
| Cloud Point: (C.°) | −23 |

### EXAMPLES 5-7

Fuel for turbine engines is specified by ASTM standard D-1655. Plant sourced oils have limited penetration in to the market for turbine fuel.

TABLE 7
Turbine Fuel

| | | 9 Parts | 7 Parts | 9 parts |
|---|---|---|---|---|
| | | Jet A: | Jet A: | Jet A |
| | | 1 Part | 3 Parts | 1 Part |
| Property- | | Fractionated | Fractionated | Fractionated |
| Measurement | ASTM D | SME- | SME- | SME- |
| Units | 1655 | Example 5 | Example 6 | Example 7 |
| Density- | 775-840 | 817.8 | 831.4 | 817.8 |
| kg/m³ | | | | |
| Viscosity | maximum | 5.471 | — | — |
| cSt @ −20° C. | 8.0 | | | |
| Freeze Point - | maximum | −42° C. | −41° C. | −40° C. |
| ° C. | −40° C. | | | |
| Net Heat of | minimum | 42.67 | 41.43 | 42.58 |
| Combustion - | 42.8 | | | |
| MJ/kg | | | | |
| Acid Value - | maximum | 0.016 | 0.028 | 0.016 |
| mgKOG/g | 0.01 | | | |

A commercially sourced soybean oil derived fatty acid methyl ester the properties of which are described in Table 2 was fractionated as described herein. The as obtained fraction analysis and the fraction analysis after processing appears in Table 8. The fractionated SME of Examples 5-7 was then blended with the Commercial Jet A fuel to yield the properties according to Table 7.

TABLE 8

| | Percent by Weight | | | |
|---|---|---|---|---|
| | Commercial | Fractionated | Fractionated | Fractionated |
| | Soy | SME | SME | SME |
| Component | Methyl Ester | Example 5 | Example 6 | Example 7 |
| methyl | 9.15 | 3.48 | 1.30 | 6.53 |
| palmitate | | | | |
| methyl stearate | 3.78 | 0.23 | 0.10 | 0.54 |
| methyl oleate | 23.52 | 28.99 | 28.17 | 28.70 |
| methyl | 55.25 | 58.12 | 60.62 | 55.95 |
| linoleate | | | | |
| methyl | 7.64 | 9.18 | 9.80 | 8.28 |
| linoleniate | | | | |
| unknown | 0.66 | 0.00 | 0 | 0 |

The fractionated soy methyl ester was blended with Jet A fuel in the ratios indicated in Table 7 yielded the properties noted. The blended fuel has demonstrates that the requirements of ASTM D-1655 are attainable with blends including SME.

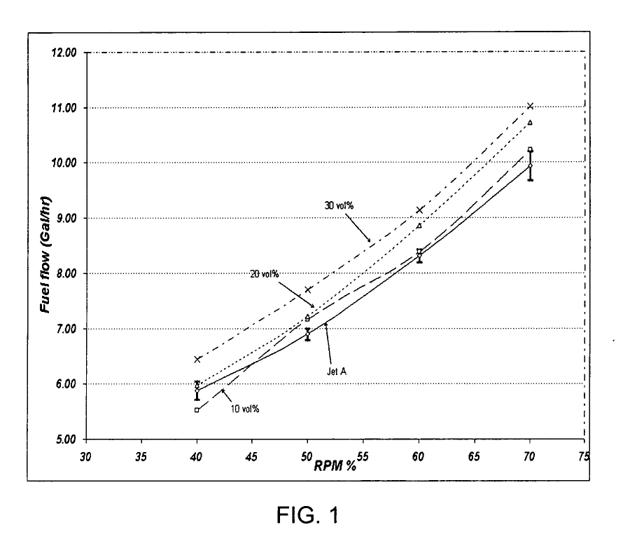

Combustion studies of SME blends with commercial Jet A show non-critical deviation from the combustion of commercial Jet A fuel. An Allison stationary 250 turbine having a relatively low compression ration of 6.2:1 was used for the combustion study. FIG. 1 shows the fuel flow rate over a power range from 40 to 70 RPM % for Jet A, and SME blends of 10%, 20% and 30% with Jet A.

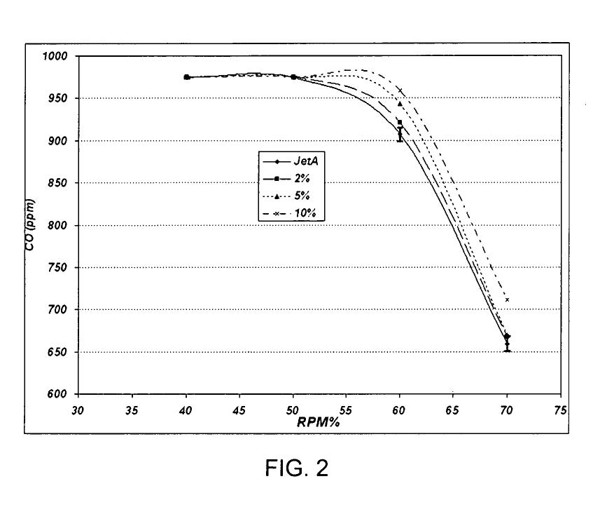

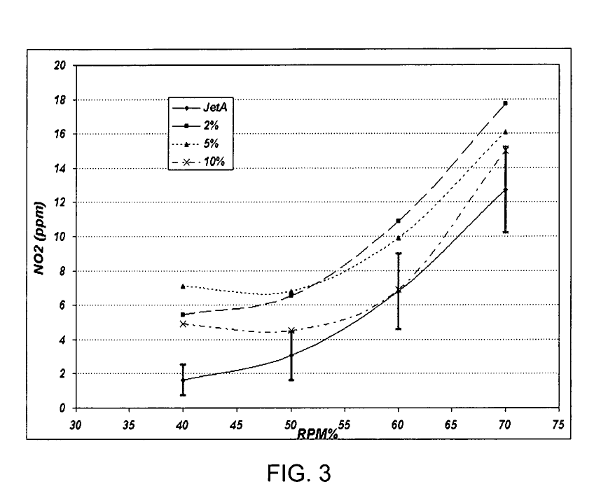

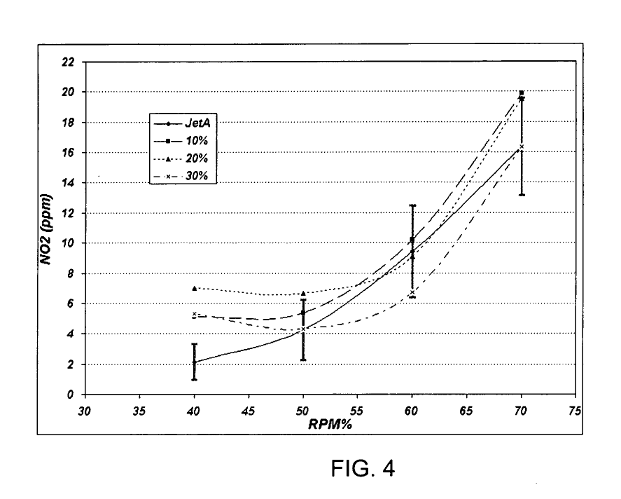

Controlled emissions for Jet A and SME blends are shown in FIG. 2 for carbon monoxide, FIG. 3 for nitrogen dioxide, and FIG. 4 for nitrogen monoxide.

While the invention has been illustrated and described in detail in the foregoing ion, such illustration and description is to be considered as exemplary and not restrictive ter, it being understood that only the preferred embodiments have been shown and d and that all changes and modifications that come within the spirit of the invention are to be protected.

1. A method for treating a fatty acid methyl ester, comprising:
mixing the fatty acid methyl ester with an amount of urea and an amount of alcohol to make (i) a urea/fatty acid methyl ester ratio of from about 0 wt/wt to about 1 wt/wt and (ii) an alcohol/fatty acid methyl ester ratio of from about 4 vol/wt to about 8 vol/wt;
heating the fatty acid methyl ester/urea/alcohol mixture to a temperature at which a homogenous mixture is obtained;
cooling the fatty acid methyl ester/urea/alcohol mixture to a temperature where a solid phase and a liquid phase are formed; and
separating the solid phase from the liquid phase.

2. The method of claim 1, wherein:
cooling the fatty acid methyl ester/urea/alcohol mixture includes cooling at a rate of from about 1 C°/min to about 15 C°/min.

3. The method of claim 1, wherein:
heating the fatty acid methyl ester/urea/alcohol mixture includes heating to a temperature of about 65 C° to about 75 C°.

4. The method of claim 1, wherein:
cooling the fatty acid methyl ester/urea/alcohol mixture includes cooling to a temperature of from about 10 C° to about 50 C°.

5. The method of claim 1, wherein:
separating the solid phase from the liquid phase includes filtering the solid phase from the liquid phase.

6. The method of claim 1, wherein:
separating the solid phase from the liquid phase includes subjecting the solid phase and the liquid phase to centrifugation to separate the solid phase from the liquid phase.

7. The method of claim 1, further comprising:
subsequent to separating the solid phase from the liquid phase, removing from about 50% to about 70% of the alcohol from the liquid phase.

8. The method of claim 7, wherein:
removing the alcohol from the liquid phase includes evaporating the alcohol from the liquid phase under a pressure less than atmospheric pressure at a temperature of from about 30 C° to about 50 C°.

9. The method of claim 1, further comprising:
subsequent to separating the solid phase from the liquid phase, washing the liquid phase with an acidic solution.

10. The method of claim 9, wherein:
the acidic solution has a pH of from about 3 to about 4 and a temperature of from about 60 C° to about 70 C°.

11. The fatty acid methyl ester prepared according to claim 1.

12. The fatty acid methyl ester prepared according to claim 9.

13. The fatty acid methyl ester prepared according to claim 10.

14. A composition comprising the fatty acid methyl ester of claim 11 wherein the composition is selected from the group consisting of a fuel, a foodstuff, nutritive compositions, pharmaceuticals, cosmetics, dermatological compositions, coatings and paints.

15. A composition comprising the fatty acid methyl ester of claim 12 wherein the composition is selected from the group consisting of a fuel, a foodstuff, nutritive compositions, pharmaceuticals, cosmetics, dermatological compositions, coatings and paints.

16. A composition comprising the fatty acid methyl ester of claim 13 wherein the composition is selected from the group consisting of a fuel, a foodstuff, nutritive compositions, pharmaceuticals, cosmetics, dermatological compositions, coatings and paints.

17. A diesel engine or turbine engine fuel comprising a fatty acid methyl ester of claim 11.

18. A diesel engine or turbine engine fuel comprising a fatty acid methyl ester of claim 12.

19. A diesel engine or turbine engine fuel comprising a fatty acid methyl ester of claim 13.

20. A method of operating an engine comprising a diesel engine or a turbine engine and further comprising using as a fuel the fatty acid methyl ester of claim 11.
